(19) United States
(12) Patent Application Publication
(10) Pub. No.: US 2008/0208130 A1
(43) Pub. Date: Aug. 28, 2008

(54) APPARATUS AND METHOD FOR HOLDING PERIPHERALLY INSERTED CATHETER LINES

(76) Inventor: Duane E. FURMAN, York, PA (US)

Correspondence Address: Saul Ewing LLP (Harrisburg);Attn: Patent Docket Clerk, Penn National Insurance Plaza, 2 North Second Street, Harrisburg, PA, 17101, US

(21) Appl. No.: 12/037,999

(22) Filed: Feb. 27, 2008

Related U.S. Application Data
(60) Provisional application No. 60/891,815, filed on Feb. 27, 2007.

(51) Int. Cl. A61M 5/31

(52) U.S. Cl. 604/164.08

(57) ABSTRACT

A protective stabilizing sleeve for catheters, such as peripheral intravenous central catheters, is provided for protecting the external portion of the catheter line. A protective device for use with peripheral intravenous catheters, the device comprising a first sleeve including a proximal open end, an opposite distal open end, and a substantially non-compressive tube of sleeve material disposed therebetween. The sleeve is preferably elastic, but remains substantially non compressive as the device is radially expanded, such as to fit over the extremity of a subject to cover a peripheral catheter inserted therein.

### CROSS REFERENCE TO RELATED APPLICATIONS

This application claims priority to co-pending U.S. provisional patent application No. 60/891,815 filed Feb. 27, 2007, which is incorporated herein by reference in its entirety.

### FIELD

The device is directed to protective devices for holding tubing connected to intravenous and internal medical devices, such as peripherally inserted central catheters (“PICC”), and for indirectly stabilizing and protecting the associated devices, such as catheters that are inserted into an extremity of a patient or subject, whether the extremity is an arm or a leg.

### BACKGROUND

Known medical devices include “peripherally inserted catheters” which are commonly inserted into the arm or leg (“extremities”) of patients or subjects for medical purposes. Within the genus of such catheters devices, there are numerous species, such as central and mid. However, for purposes herein, all peripheral inserted catheter devices suitable for insertion though an extremity are collectively referred to as “PICC” or “PICC lines” devices. The PICC line is generally a long, soft plastic tube that can be, for example, inserted into a large vein near the bend in the arm (antecubital fossa), then threaded into the large blood vessel near the heart. In such an embodiment, the PICC line allows for medication or fluids to be put directly into the blood stream and also allows for collection of blood samples. In other embodiments, similar catheters may be inserted into other patient extremities for treatment, diagnostic and/or surgical purposes, and may also derive benefit. In any case, once the PICC line is inserted, there is an external amount of tubing, which may range from 5-15 centimeters, that remains outside the patient's body. It is this tubing that the device is primarily designed to support and protect.

Importantly, PICC devices and associated lines and tubes are not intravenous needles, and the device is distinguishable from devices known for securing intravenous needles and associated tubes and lines. The basic structure of intravenous needles and associated tubes connected thereto is that a needle hub is connected to one end of a delivery tube, the opposite end of the delivery tube being connected to a reservoir supply of liquid. The insertion end of the needle is inserted through the patient's skin and just far enough to breach a targeted vein located just beneath the skin. Next, the delivery tube is secured to the hub end of the needle. Often, the tube and/or needle hub are secured to the patient's skin with adhesive tape in order to prevent unintended removal of the needle, such as if the tube is pulled by patient movement. In such arrangements, the needle is exposed except for the portion inserted into the vein, and thus the exposed needle and insertion site may be susceptible to contamination. Over the years others have invented stabilizing apparatus for IV needles and the associated tube as well for holding and protecting the IV needles. For example, devices are known for positioning and securing an IV needle and associated tubing. Intravenous needles are well known as a means to deliver liquids into a patient's vein. Representative of some of these earlier attempts are the devices disclosed in the following issued U.S. Pat. No. 4,591,356 to Christie; U.S. Pat. No. 4,822,342 to Brawner; U.S. Pat. No. 4,898,587 to Mera; U.S. Pat. No. 3,900,026 to Wagner; U.S. Pat. No. 4,870,976 to Denny; and U.S. Pat. No. 5,188,608 to Fritts. Descriptions of those patents are provided herein simply because such devices are thought to be the closest, though non-analogous, art known to the inventor at this time.

Christie discloses an intravenous needle-stabilizing band, which wraps around the arm of the patient. The exterior surface of the band includes opposing flaps which open to permit passage over the tube part of the needle and means to secure the tube in place directly onto the exterior surface of the band. Brawner discloses a prepared tape for a body inserted tube, such as an intravenous needle. The device includes a multi-layer arrangement typically involving two separate portions for clamping the hub of the needle and for securing the delivery tube. Mera discloses an intravenous line stabilizing device configured as a two-part clamping arrangement which has a specifically contoured shape to provide a clearance slot for the needle hub and a channel for the tube. Once the base plate is secured directly to the patient's arm the needle hub and tube are laid into position and a top cover or clamping plate is placed on top. A second clamp is used to secure the tube to the arm of the patient. Wagner discloses a somewhat complicated device for holding and protecting intravenous injection needles. As is illustrated in FIGS. 1 and 2, there is a cap member 15 which is applied directly to the arm and has an angled neck portion 17 for securing the needle hub and tube in an inclined and upwardly elevated orientation. Denny discloses a very elaborate intravenous injection shield assembly which includes a protective shield member made of a rigid transparent plastic. The assembly further includes strap members for holding the assembly in a predetermined position on a limb and defines a clearance space which is suitable to receive an intravenous needle and tubing. The disclosed design is somewhat complex with a number of straps and ties which must be completed as well as having substantial size and weight and clearly not providing the type of device which could be easily applied and removed. Fritts discloses a two-layered sleeve having an outer fabric layer and an inner of porous gauze material with a non-stick surface. The outer layer is free of any exterior structural member that could be caught or bumped or hooked on an object thereby pulling the needle loose.

In contrast, a PICC device is inserted entirely through the skin to allow a catheter to penetrate through and travel within a target vein or artery, such as to the heart for infusion of medications, and alternatively for conducting an internal medical procedure such as angioplasty and other diagnostic and/or surgical procedures. The PICC device may remain in the patient for extended periods of time, such as up to 12 weeks as in the case of a medication infusion device. Upon installation of a PICC line and device, it is common practice to leave a length of excess external tubing extending from the insertion site, such as to enable easy connection of infusion lines or diagnostic and surgical devices. This excess catheter tubing commonly is either taped to the extremity, or sometimes folded into a primitive sleeve such as a cut-off sock. Obviously, such a primitive can place the excess tubing at risk for migrating out of insertion site, trauma, damage, contamination, and various other catastrophes.

None of the prior art discussed herein addresses the unique aspects of PICC lines and PICC devices, nor the unique needs for stabilizing, protecting, supporting, and maintaining the reasonable cleanliness and/or sterility of such devices once inserted into a patient, and especially during extended periods of time, up to months of use. While some of the structural concepts of the IV stabilizing references relate to stabilizing the needle and tube and while some references disclose partial covering of the needle, none are believed to anticipate the present device. Further, the simplicity of the device and the additional enhancements provided would not have been obvious in view of the above-listed references.

### SUMMARY

A protective stabilizing sleeve is provided for PICC lines, and particularly for stabilizing and maintaining acceptable levels of cleanliness and/or sterility for excess PICC tubing extending from an insertion site on a patient extremity. The device according to one embodiment comprises a first sleeve comprised of a non-compressive material, the sleeve having proximal and distal ends and an exterior surface therebetween, the sleeve being arranged to cover external portion of a PICC tube that is connected to a PICC device inserted in a patient. The first sleeve is comprised of material that is supportive for retaining a length of tubing protruding from an insertion site on a patient's extremity without exerting compressive forces sufficient to interfere with fluid flow through the tubing or to cause patient discomfort. The first sleeve is preferably comprised of material that is exhibits elasticity without exhibiting an increase in compressive force upon the extremity as the sleeve diameter is increased. The sleeve preferably comprises antimicrobial properties, whether imparted by selection of the sleeve material, or by application of an antimicrobial agent to the sleeve.

The protective stabilizing sleeve optionally includes a second sleeve of non-compressive material surrounded by the first sleeve and positioned for placement over the patient's extremity at or near the insertion site for securing the sleeve around the extremity of the patient and stabilizing the external PICC tube.

Either sleeve may optionally be comprised of an antimicrobial material. Such an antimicrobial aspect will decrease the risk of skin bacteria, fungal components and moisture from influencing the PICC insertion site and underlying dressing. For example, a sleeve may be constructed of an antimicrobial microfiber material having expandability, but with little or no compression, to secure the extraneous tubing. Such a microfiber material would be selected to expand when placed on an extremity, but not to unduly compress the extremity or the tubing. This aspect is especially important given the need for patient comfort in extended applications, such as infusion of antibiotics over a several week period of time in a patient suffering a resistant bacterial infection. For example, the sleeve may be air (e.g., oxygen) and water vapor permeable. It may also contain a biocide, such as an antibiotic that imparts MRSA resistance to the device.

Accordingly, one object of the present invention is to provide an improved protective stabilizing sleeve for a PICC line and associated components. A second objective is to provide an antimicrobial sleeve to maintain an acceptable level of sterility at and around a PICC insertion site and associated external components such at PICC tubing, without exerting excessive compressive force that can lead to patient discomfort, insertion site irritation, skin irritation, poor circulation, or any other undesirable effect commonly encountered by current patients who have PICC lines inserted for extended periods of time.

Related object and advantages of the present invention will be apparent from the following description.

### BRIEF DESCRIPTION OF THE DRAWINGS

FIG. 1 is a top plan view of an anatomically sized, skin-contacting protective stabilizing sleeve for an intravenous needle applied to a patient's arm according to a typical embodiment of the present invention.

FIG. 2 is a top plan view of a second embodiment of a protective stabilizing sleeve according to the present invention.

FIG. 3 is a side elevational view in full section illustrating the material lamination construction for the various embodiments of the present invention.

### DESCRIPTION OF A PREFERRED EMBODIMENT

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

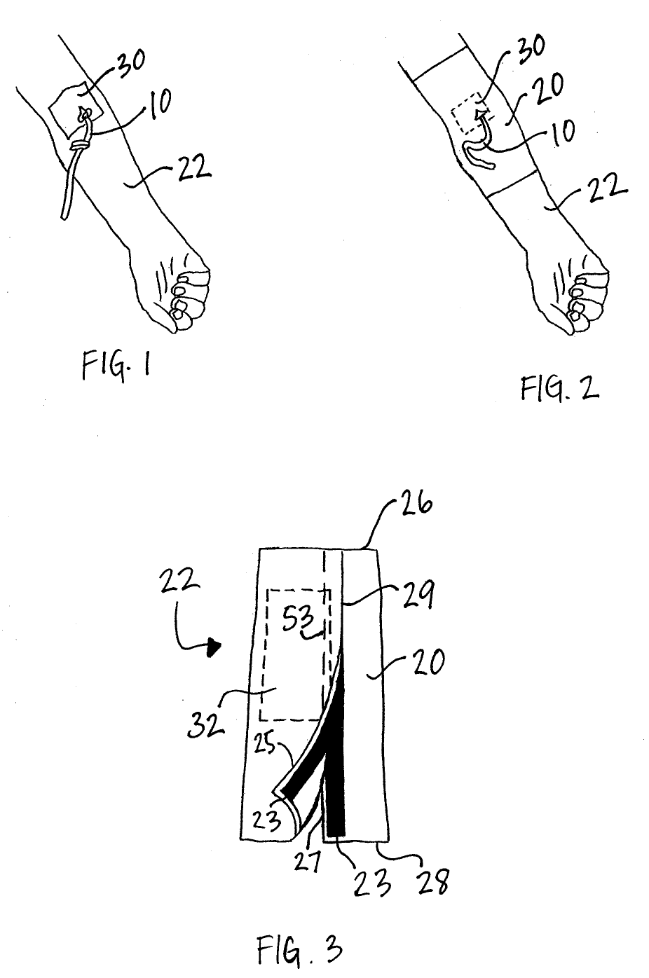

Referring to FIG. 1 there is illustrated a PICC line 10 inserted into a patient's arm 22, with no sleeve 20 provided. The line 10 connects to a PICC device which is inserted into a vein in the arm 22 of the patient, with an external portion of the PICC line 10 remaining extending outside of the insertion site. The insertion site is covered by sterile gauze 30.

Referring to FIG. 2, there is illustrated in partial cross section a PICC line 10 inserted into a patient's arm 22, with a sleeve 20 provided. As in FIG. 1, the line 10 connects to a PICC device which is inserted into a vein in the arm 22 of the patient. However, in FIG. 2, the external portion of the PICC line 10 remaining extending outside of the insertion site is covered by the sleeve 20. Again, the insertion site is covered by sterile gauze 30. The sleeve 20 thus provides both protection for the insertion site, as well as protection of the external portion of the PICC line 10.

As previously discussed, there are two important concerns in the use of PICC lines. One concern is the cleanliness of the site and the desire to keep the insertion site free from any type of dirt, debris or contaminants. The other concern relates to protecting, supporting, holding and stabilizing the external portion of the PICC line 10 and maintaining acceptable cleanliness of the external portion of the line 10. Stabilizing and holding of the external PICC line 10 also helps to guard against the line 10 being caught or pulled and as a result pulling the PICC device loose or otherwise damaging the PICC line, device and/or the patient.

As further shown in FIG. 3, in the present invention the first sleeve 20 is configured and sized to fit around the arm 22 or other extremity of the patient in a substantially non-compressive manner, and to completely cover the insertion site and dressing 30. For purposes of this application, “substantially non-compressive” means that the device exerts the minimal force necessary to hold and support any protruding equipment associated with an inserted peripheral catheter, but less force than would interfere with the functional use of the inserted device, and also less force than would result in discomfort to the patient or subject wearing the device. Ideally, a substantially non-compressive device does not significantly impede venous blood flow while in use. Also, it is desirable for the device to be expandable in at least the radial direction (i.e. perpendicular to a longitudinal central axis of the sleeve 20), without significantly increasing the compressive forces exerted by the tube in the opposite direction (i.e. towards the skin of a user, whose arm would be aligned along a longitudinal central axis of the device). Optionally, as shown, the sleeve 20 may have a slightly tapering design in recognition that the arm 22, such as from the wrist to the elbow, has a slight increase in diameter size. In one embodiment, the sleeve may be continuous from first proximal open end 26 to opposite distal end 28. The sleeve 20 is made from material that possesses elastic properties in at least the radial orientation, yet remains minimally compressive. Thus, the sleeve 20 is expandable so as to allow fitting over a patient's extremity and covering of the line 10 without unduly compressing the extremity or line 10.

Optionally, in another example, the sleeve 20 may be split, such as by an open seam 29 extending the full length of the sleeve between the proximal open end 26 and distal open end 28. In this embodiment, the circumferential size of the sleeve is large enough to provide an area of overlap to allow the seam 29 to be closed by fastening means, even for large patients. Sleeve edges 25 and 27 which create seam 29 are not otherwise connected except at their ends. For example, each end 26, 28 of the sleeve 20 may include fastening means 23 at the ends 26, 28, center, and/or at any position along the edges 25, 27 so as to provide a closed sleeve 20. The fastening means 23 may be any means known in the art, such as buttons, snaps, hooks, zippers, hook and loop fasteners such as hook and loop type fasteners (for example VELCRO® brand hook and loop fasteners comprising a mating combination of hook-like and loop-like projections) and combinations thereof.

To prevent undesired levels of compression pressure on extremity 22 exerted by sleeve 20, the sleeve maybe provided in distinct sizes, and may optionally be adjustable such as in the open seam example previously discussed herein. In an open seam embodiment, the circumferential size can be adjusted by the fastening means. Another method to reduce the compressive pressure of sleeve to put ends 26, 28 closer together along the arm 22. This arrangement of pushing the ends closer together provides extra sleeve material in the region of the insertion site 30, and provides surplus material so that there is little or no pressure on site 30.

In yet another embodiment, the sleeve 20 may be a multiple layer material, or may be comprised of several nested sleeves of varying materials and other characteristics. For example, an inner layer 32 may include a non-stick porous gauze material, antimicrobial material, pharmaceutically active material, or other desirable physical and chemical characteristics for the healing arts. The layer 32 may be partial, or may be a second sleeve that covers the entire inner surface of the sleeve 20. For example, the layer 32 may include a suitable porous gauze layer material such as that used with non-stick bandages that allow the site to remain sterile while providing absorption of any blood or other body fluid without sticking to any opening or wound.

One advantage of the design of sleeve 20 compared to other medical devices and concepts is the fact that the entire sleeve 20 is preferably completely free of any tube tie-downs, clamps, brackets, holders, tape layers, raised portions or other protuberances, or structural elements. In this embodiment, the sleeve surfaces are smooth and clean and there is nothing on the outer sleeve 20 surface to get bumped, hit, caught, pulled or jarred, and nothing to come loose. The sleeve material is flexible and lightweight and is quickly and easily put around the arm, after the needle is inserted into a vein, with a minimum of effort and discomfort to the patient. Part of the novelty of this embodiment is the recognition of simplicity and a realization that the earlier complex designs are not necessary or desirable for use with PICC lines.

It is also to be understood that in lieu of fabricating sleeve 20 out of a fabric which can be washed and reused, one could use a disposable material similar to the materials currently used for disposable gowns and masks. This same disposable paper material could be used for the design of sleeve 20. When a disposable paper or other disposable material is used the porous gauze layer would not have to be provided as a removable element but could simply be bonded or sewn directly to the outer sleeve layer as it would be disposed of along with the sleeve. With a disposable material the fasteners, such as VELCRO strips, could be adhesively bonded to the surfaces and as would be contemplated, the various VELCRO fastener strips can be replaced by adhesive means or mechanical fasteners such as snaps. It is also envisioned that a drawstring style of securement could be used for sleeve 20. Finally, the VELCRO strips which are used with the fabric layers are secured in place by either the use of a bonding adhesive or are sewn to the fabric material at the appropriate locations.

Additional Points Regarding the Device:

The PICC line sleeve is designed to secure the remaining tubing that is external of the insertion site in the extremity. The insertion point of the catheter typically already has a sterile securing dressing which this device does not replace.

Sleeve may be constructed of a woven antimicrobial microfiber material to provide desired radial elasticity and expandability, without a corresponding increase in radially compressive force, to secure the extraneous external tubing. In this example, the sleeve 20 is an open-ended tubular device. Optionally, the microfiber may be impregnated with antimicrobial agents to prevent or retard the growth of undesirable organisms, such as by way of non-limiting example, fungus, microbes, germs, bacteria, viruses. Any antimicrobial technology can be utilized so long as it is compatible with the sleeve material. By way of non-limiting example, for textile sleeve materials, whether natural or synthetic fibers, antimicrobial technologies can include metal ions (silver, copper, etc.), known antimicrobial chemicals, antiviral chemicals, and combinations thereof. without adversely affecting the desired properties of the sleeve 20. Such antimicrobial aspects will decrease the risk of skin bacteria, fungal components and moisture from influencing the PICC insertion site and underlying dressing.

The device will provide protective measures regarding the excess catheter tubing, which now commonly is either taped to the extremity or folded into a “cut-off” sock by a patient or practitioner. Obviously, such a primitive solution can place the excess tubing at risk for migrating out of insertion site, trauma and various other catastrophes. It is primarily designed to protect the excess catheter tubing while not adversely influencing the internal extremity tubing.

The device comprises a sleeve with proximal and distal openings, and may be a one-piece continuous material. It may come in varying sizes; S-M-L-XL, and secured by the expandability of the antimicrobial microfiber material. Optionally, it may have a seam and use straps, hooks or any additional securing application device.

A PICC line is a “peripherally inserted central catheter.” The PICC line is a long, soft plastic tube that is placed into a large vein near the bend in the arm (antecubital fossa), then threaded into the large blood vessel near the heart. It allows for medication or fluids to be put directly into the blood stream and also allows for collection of blood samples. Once the PICC line is inserted, there is an external amount of tubing, which may range from 5-15 centimeters. It is this tubing the device is specifically designed to protect.

The device is designed for PICC lines and not intravenous needle securing devices such as mentioned in Christie and Fritts patents. Those devices are specific for IV needle insertions, not PICC lines. As used herein, intravenous needles, per se, when inserted into a patient's vein are not intended to be a PICC line.

Regarding drawings, notice the arm without the sleeve; the tubing simply hangs from the insertion site under the sterile dressing (gauze). The arm with the sleeve is safely secured under the device and kept clean as well as safe. An additional point, if of importance, is that when it is time for the patient to infuse the medication, the sleeve can easily be rolled back or removed prior to infusion, then reapplied following completion of the infusion.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A device for use with peripheral intravenous catheters, the device comprising a first sleeve including a proximal open end, an opposite distal open end, and a tube of substantially non-compressive sleeve material disposed there between.

2. The device of claim 1, wherein the proximal open end of the first sleeve is larger than the opposite distal open end of the first sleeve.

3. The device of claim 2, wherein the continuous tube of substantially non-compressive sleeve material is radially expandable, and remains substantially non-compressive when the tube is radially expanded.

4. The device of claim 3, wherein the continuous tube of sleeve material remains substantially non-compressive when placed over the extremity of a mammal to cover a peripherally inserted catheter inserted in the extremity.

5. The device of claim 4, wherein the sleeve material comprises at least one antimicrobial agent.

6. The device of claim 5, wherein the sleeve material comprises microfibers.

7. The device of claim 6, wherein the sleeve material comprises a woven textile.

8. The device of claim 4, wherein the inserted catheter is a peripheral intravenous central catheter.

9. The device of claim 1, further comprising a second sleeve nested within the first sleeve.

10. The device of claim 1, wherein the tube of substantially non-compressive sleeve material includes at least one open seam extending from the proximal open end to the distal open end.

11. A kit for use with peripheral intravenous catheters, the kit comprising: at least one of a catheter, catheter tubing, needle, bandage, antiseptic, or gauze; and at least one device, the device comprising a first sleeve including a proximal open end, an opposite distal open end, and a tube of substantially non-compressive sleeve material disposed therebetween.

12. The kit of claim 11, wherein the proximal open end of the first sleeve is larger than the opposite distal open end of the first sleeve.

13. The device of claim 12, wherein the substantially non-compressive tube of sleeve material is radially expandable as a result of at least one elastic property of the material, and wherein the device remains substantially non-compressive even when radially expanded.

14. The device of claim 13, wherein the device is substantially non-compressive when placed over the extremity of a mammal so as to cover a peripherally inserted catheter site of the extremity.

15. The device of claim 14, wherein the sleeve material comprises at least one antimicrobial agent.

16. The device of claim 15, wherein the sleeve material comprises microfibers.

17. The device of claim 16, wherein the sleeve material comprises a woven textile.

18. The device of claim 14, wherein the catheter is a peripheral intravenous central catheter.

19. The device of claim 11, wherein the device further comprises a second sleeve nested within the first sleeve.

20. The device of claim 11, wherein the tube of sleeve material includes at least one open seam extending from the proximal open end to the distal open end.
